(19) United States
(12) Patent Application Publication
(10) Pub. No.: US 2025/0127755 A1
(43) Pub. Date: Apr. 24, 2025

(54) PHARMACOLOGICAL DEPLETION OF HEME FOR THE TREATMENT OF MYELODYSPLASTIC SYNDROME

(71) Applicant: THE SCRIPPS RESEARCH INSTITUTE, La Jolla, CA (US)

(72) Inventors: Daniel Kremer, La Jolla, CA (US); Sida Shao; Peter G. Schultz, La Jolla, CA (US); Michael J. Bollong, San Diego, CA (US)

(21) Appl. No.: 18/517,049

(22) PCT Filed: May 16, 2022
(86) PCT No.: PCT/US2022/072339

§ 371 Date: Nov. 22, 2023

Related U.S. Application Data
(60) Provisional application No. 63/202,036, filed on May 24, 2021.

(51) Int. Cl. A61K 31/357; A61K 31/404; A61K 31/4178; A61K 31/4196; A61K 31/436; A61K 31/438; A61K 31/444; A61K 31/4525; A61K 31/4545; A61K 31/4709; A61K 31/496; A61K 31/506; A61K 31/5377; A61K 31/541

(52) U.S. Cl. CPC A61K 31/357; A61K 31/404; A61K 31/4178; A61K 31/4196; A61K 31/436; A61K 31/438; A61K 31/444; A61K 31/4525; A61K 31/4545; A61K 31/4709; A61K 31/496; A61K 31/506; A61K 31/5377; A61K 31/541

(57) ABSTRACT

Disclosed herein are methods for using an antimalarial endoperoxide compound, such as artemisinin, in treating a subject suffering from myelodysplastic syndromes (MDS), and hi slowing or preventing the progression of MDS in the subject to development of acute myeloid leukemia (AML).

This application claims the benefit of priority to U.S. Provisional Patent Application No. 63/202,036, filed on May 24, 2021, which application is incorporated in its entirety as if fully set forth herein.

### BACKGROUND

Myelodysplastic syndromes (MDS) are clonal stem-cell disorders characterized by ineffective hemopoiesis leading to cytopenias and increased blast cell count. About one-third of MDS patients subsequently progress to acute myeloid leukemia (AML). Advances over the past few decades have uncovered the genetic cause of MDS: it is now understood that somatic mutations (e.g., TET2, RUNX1, DNMT3A) in hematopoietic progenitor cells (HPCs) lead to aberrant regulation of hemopoiesis. To date, the only FDA-approved medicines for treating MDS are repurposed AML drugs, including Azacitidine and Decitabine, which promote epigenetic changes in cells leading to cytotoxicity.

Anemia affects the majority of MDS patients, leading to fatigue, poor quality of life and destabilization of underlying cardiovascular disease. As a result, chronic blood transfusions are typically necessary for MDS patients. However, iron overloading due to transfusion dependence is also associated with decreased survival and increased progression to acute myeloid leukemia.

The last U.S. FDA approval of a drug for MDS occurred in 2006 with the approval of lenalidomide for low- or intermediate-risk MDS with 5q deletion. Before that, azacitidine and decitabine were approved in 2004 and 2005, respectively. Although these drugs were breakthroughs and have helped extend the lives of the MDS patient population, they are not curative for most patients, for whom relapsed disease is almost certain. The only known cure for MDS is an allogeneic hematopoietic stem cell transplant, but its use is limited by the older age and attendant comorbidities of typical MDS patients.

### SUMMARY

The present disclosure overcomes these shortcomings of MDS therapies by providing, in an embodiment, a method for treating a myelodysplastic syndrome (MDS) in a subject suffering therefrom. The method comprises administering to the subject a therapeutically effective amount of an antimalarial endoperoxide compound or a pharmaceutically acceptable salt thereof.

In another embodiment, the present disclosure provides a method for slowing or preventing the development of leukemia in a subject who suffers from a myelodysplastic syndrome (MDS). The method comprises administering to the subject a therapeutically effective amount of an antimalarial endoperoxide compound or a pharmaceutically acceptable salt thereof.

The present disclosure also provides, in an additional embodiment, an antimalarial endoperoxide compound or a pharmaceutically acceptable salt thereof for treating a myelodysplastic syndrome (MDS) in a subject suffering therefrom.

In still another embodiment, the present disclosure provides an antimalarial endoperoxide compound or a pharmaceutically acceptable salt thereof for slowing or preventing the development of leukemia in a subject who suffers from a myelodysplastic syndrome (MDS).

### BRIEF DESCRIPTION OF THE DRAWINGS

FIG. 1. Schematic depicting the differentiation roadblock observed in myelodysplastic syndromes, and the effect of endoperoxide compounds on the same.

FIG. 2. High throughput multi-parametric flow cytometry-based assay for identifying inhibitors of MDS differentiation using MDS-L cells.

FIG. 3. Exemplary antimalarial endoperoxide compounds are efficacious differentiation-inducing agents in MDS-L cells.

FIG. 4A-FIG. 4C. Heme depletion induces terminal differentiation of MDS-L cells. A: Relative induction of GlyA positive MDS-L cells in response to treatment for 96 hours with the indicated concentrations of NMPP. B: Heme levels of MDS-L cells treated with the indicated compounds. C: Relative induction of GlyA positive cells after 96-hour treatment with artemisone and hemin.

### DETAILED DESCRIPTION

The present disclosure relates, in part, to repurposed drugs that correct the differentiation roadblock observed in MDS disease, allowing for the appropriate replenishment of the downstream blood lineages, an effect unachievable by standard chemotherapy drugs. More specifically, the present disclosure relates to therapeutic uses of known antimalarial medications as efficacious inducers of differentiation in MDS blast-like cells. Though the present disclosure is not bound by any particular theory, it is believed that induction occurs through a mechanism involving heme depletion.

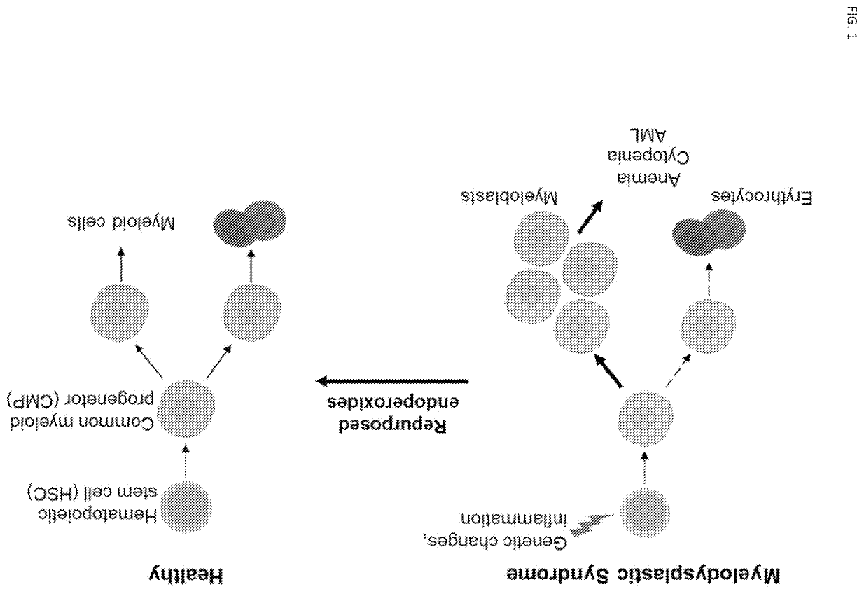

Ineffective erythropoiesis in MDS patients primarily results from aberrant regulation of terminal erythrocyte differentiation and maturation. A characteristic repertoire of MDS-associated mutations in HPCs induce a “differentiation roadblock” at later stages of the hierarchical differentiation tree. Re-stimulation of terminal erythrocyte differentiation and maturation in response to erythropoiesis-inducing stimuli (such as erythropoietin (EPO)) in erythrocyte progenitors underlies a functional cure for MDS. To this end, the present disclosure identifies repurposed drugs from high-throughput phenotypic screening for the functional rescue of erythropoiesis in patients with MDS (FIG. 1).

### Definitions

As used herein, and unless otherwise clear from context or specified to the contrary, the term “compound” is inclusive in that it encompasses a compound or a pharmaceutically acceptable salt thereof.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In various embodiments, the terms refer to minimizing the spread or worsening of the disease resulting from the administration of one or more prophylactic or therapeutic compounds described herein to a patient with such a disease.

The terms “prevent,” “preventing,” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Methods of Use

In some embodiments, the present disclosure provides a method for treating a myelodysplastic syndrome (MDS) in a subject suffering therefrom. The method comprises administering to the subject a therapeutically effective amount of an antimalarial endoperoxide compound or a pharmaceutically acceptable salt thereof.

As illustrated by the appended examples, screening hits identified endoperoxide compounds that, although heretofore unknown to possess any therapeutic activity in an MDS context, have demonstrated therapeutic but pathologically unrelated antimalarial uses. Thus, in accordance with some embodiments, the endoperoxide compound is the naturally-occurring artemisinin, derived from the plant Artemisia annua, or any known artemisinin derivatives, including semisynthetic derivatives. Non-limiting examples of these compounds, per various embodiments, are shown in Table 1.

TABLE 1
Artemisinin and some its known derivatives.

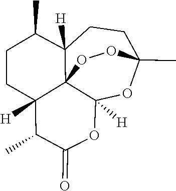 [SMILES: C[C@@H]1CC[C@H]2[C@@H](C)C(=O)O[C@@H]3O[C@@]4(C)CC[C@@H]1[C@]32OO4]

artemisinin

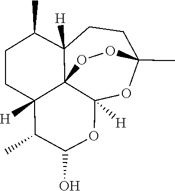

dihydroartemisinin

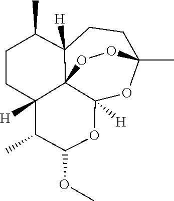

artemether

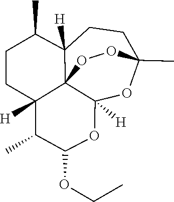

arteether

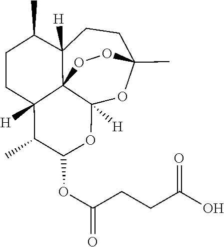

artesunate

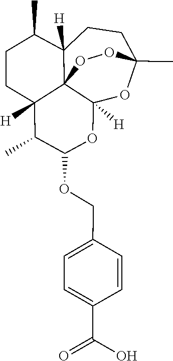

artelinate

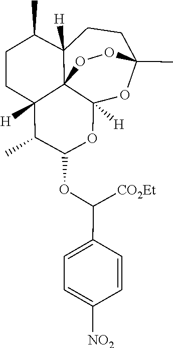

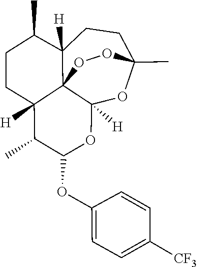

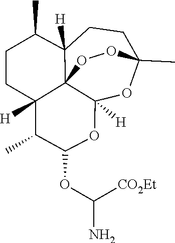

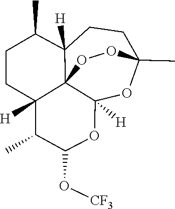

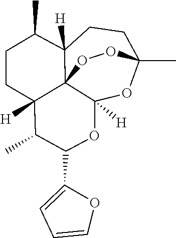

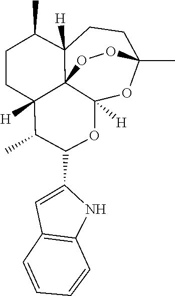

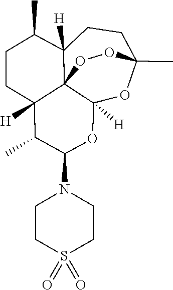 [SMILES: C[C@H]1[C@H](N2CCS(=O)(=O)CC2)O[C@@H]2O[C@@]3(C)CC[C@H]4[C@H](C)CC[C@@H]1[C@@]24OO3]

Artemisone

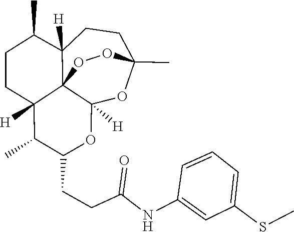

3-artesanilide

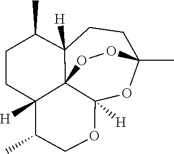 [SMILES: C[C@@H]1CC[C@H]2[C@@H](C)CO[C@@H]3O[C@@]4(C)CC[C@@H]1[C@]32OO4]

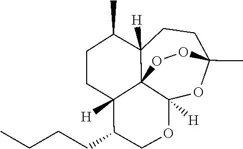 [SMILES: CCCC[C@H]1CO[C@@H]2O[C@@]3(C)CC[C@H]4[C@H](C)CC[C@@H]1[C@@]24OO3]

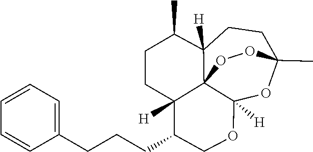 [SMILES: C[C@@H]1CC[C@H]2[C@@H](CCCc3ccccc3)CO[C@@H]3O[C@@]4(C)CC[C@@H]1[C@]32OO4]

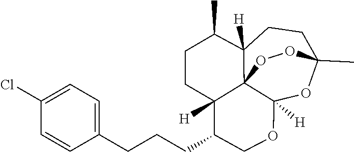 [SMILES: C[C@@H]1CC[C@H]2[C@@H](CCCc3ccc(Cl)cc3)CO[C@@H]3O[C@@]4(C)CC[C@@H]1[C@]32OO4]

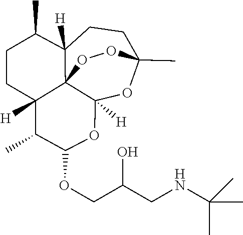

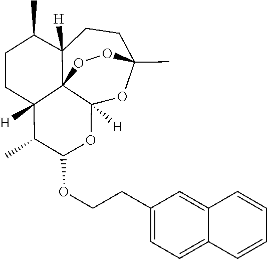

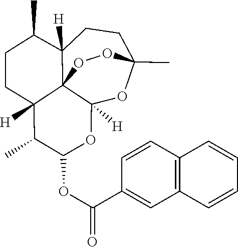

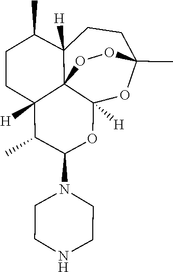 [SMILES: C[C@H]1[C@H](N2CCNCC2)O[C@@H]2O[C@@]3(C)CC[C@H]4[C@H](C)CC[C@@H]1[C@@]24OO3]

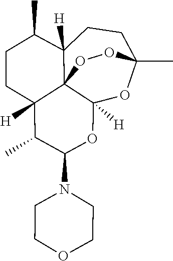 [SMILES: C[C@H]1[C@H](N2CCOCC2)O[C@@H]2O[C@@]3(C)CC[C@H]4[C@H](C)CC[C@@H]1[C@@]24OO3]

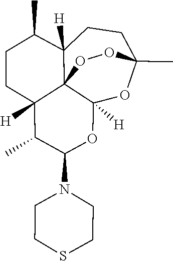 [SMILES: C[C@H]1[C@H](N2CCSCC2)O[C@@H]2O[C@@]3(C)CC[C@H]4[C@H](C)CC[C@@H]1[C@@]24OO3]

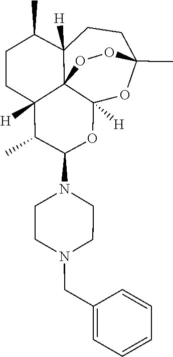 [SMILES: C[C@H]1[C@H](N2CCN(Cc3ccccc3)CC2)O[C@@H]2O[C@@]3(C)CC[C@H]4[C@H](C)CC[C@@H]1[C@@]24OO3]

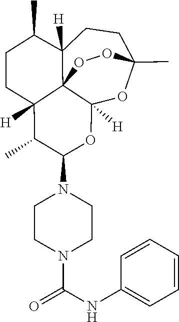 [SMILES: C[C@H]1[C@H](N2CCN(C(=O)Nc3ccccc3)CC2)O[C@@H]2O[C@@]3(C)CC[C@H]4[C@H](C)CC[C@@H]1[C@@]24OO3]

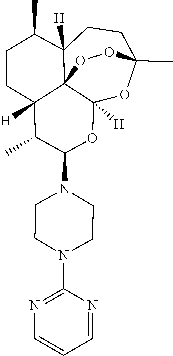 [SMILES: C[C@H]1[C@H](N2CCN(c3ncccn3)CC2)O[C@@H]2O[C@@]3(C)CC[C@H]4[C@H](C)CC[C@@H]1[C@@]24OO3]

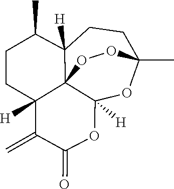 [SMILES: C=C1C(=O)O[C@@H]2O[C@@]3(C)CC[C@H]4[C@H](C)CC[C@@H]1[C@@]24OO3]

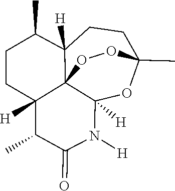 [SMILES: C[C@@H]1CC[C@H]2[C@@H](C)C(=O)N[C@@H]3O[C@@]4(C)CC[C@@H]1[C@]32OO4]

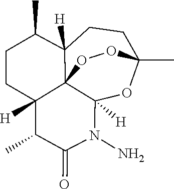 [SMILES: C[C@@H]1CC[C@H]2[C@@H](C)C(=O)N(N)[C@@H]3O[C@@]4(C)CC[C@@H]1[C@@]23OO4]

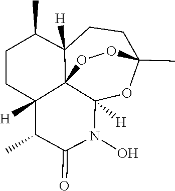 [SMILES: C[C@@H]1CC[C@H]2[C@@H](C)C(=O)N(O)[C@@H]3O[C@@]4(C)CC[C@@H]1[C@@]23OO4]

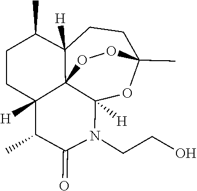 [SMILES: C[C@@H]1CC[C@H]2[C@@H](C)C(=O)N(CCO)[C@@H]3O[C@@]4(C)CC[C@@H]1[C@@]23OO4]

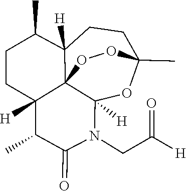 [SMILES: C[C@@H]1CC[C@H]2[C@@H](C)C(=O)N(CC=O)[C@@H]3O[C@@]4(C)CC[C@@H]1[C@@]23OO4]

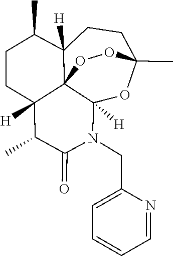 [SMILES: C[C@@H]1CC[C@H]2[C@@H](C)C(=O)N(Cc3ccccn3)[C@@H]3O[C@@]4(C)CC[C@@H]1[C@@]23OO4]

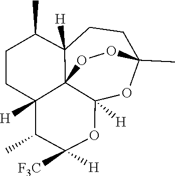 [SMILES: C[C@H]1[C@H](C(F)(F)F)O[C@@H]2O[C@@]3(C)CC[C@H]4[C@H](C)CC[C@@H]1[C@@]24OO3]

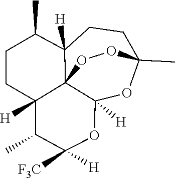 [SMILES: C[C@H]1[C@H](C(F)(F)F)O[C@@H]2O[C@@]3(C)CC[C@H]4[C@H](C)CC[C@@H]1[C@@]24OO3]

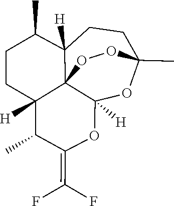 [SMILES: C[C@@H]1CC[C@H]2[C@@H](C)C(=C(F)F)O[C@@H]3O[C@@]4(C)CC[C@@H]1[C@]32OO4]

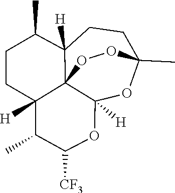

In other embodiments, the antimalarial endoperoxide compound is one that contains a 1,2,4-trioxane ring. Examples of such compounds bearing this structural feature, in accordance with various embodiments, are known in the art and illustrated in Table 2.

TABLE 2
Specific examples of antimalarial 1,2,4-trioxane compounds.

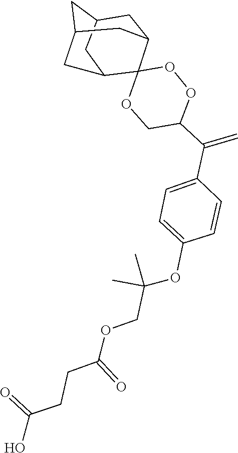 [SMILES: C=C(c1ccc(OC(C)(C)COC(=O)CCC(=O)O)cc1)C1COC2(OO1)C1CC3CC(C1)CC2C3]

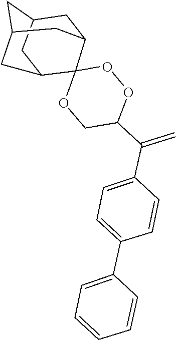 [SMILES: C=C(c1ccc(-c2ccccc2)cc1)C1COC2(OO1)C1CC3CC(C1)CC2C3]

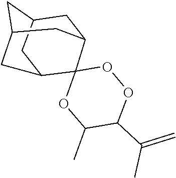 [SMILES: C=C(C)C1OOC2(OC1C)C1CC3CC(C1)CC2C3]

Another subclass of an antimalarial endoperoxide compound useful in the presently disclosed methods is a compound containing a 1,2,4-trioxololane ring. In various embodiments, exemplary compounds include those presented in Table 3.

TABLE 3
Examples of 1,2,4-trioxolane compounds.

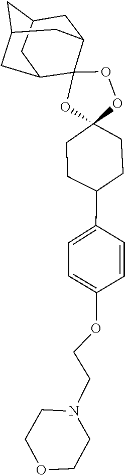

artefenomel

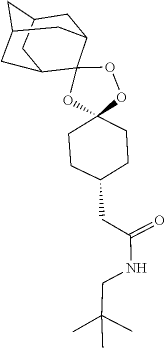

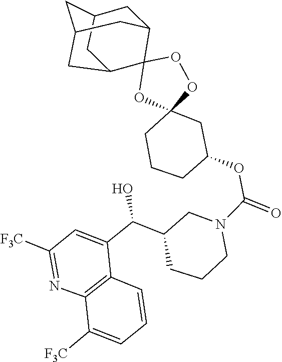

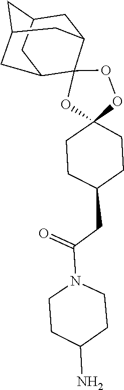

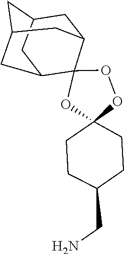

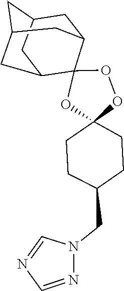

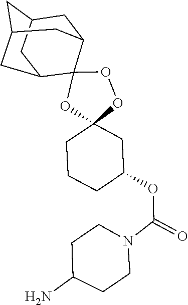

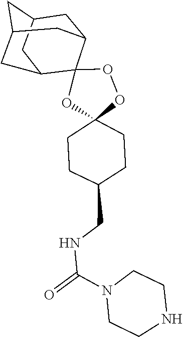

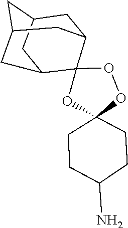

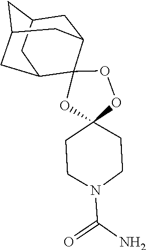

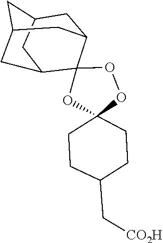

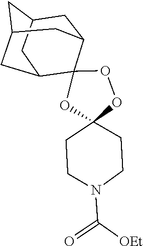

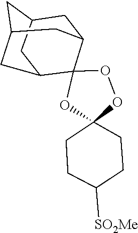

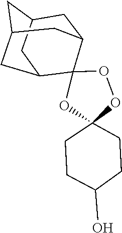

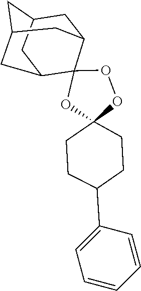

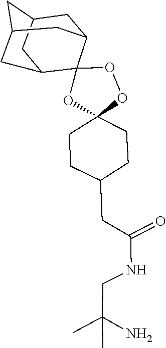

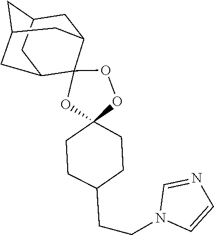

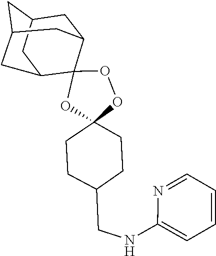

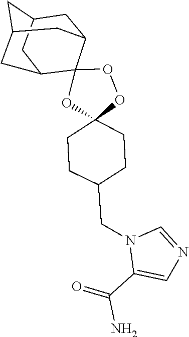

In still additional embodiments, the antimalarial endoperoxide compound is one that contains a 1,2,4,5-tetraoxane ring. Non-limiting examples of such a compound, per various embodiments, are shown in Table 4.

TABLE 4
Examples of 1,2,4,5-tetraoxane compounds.

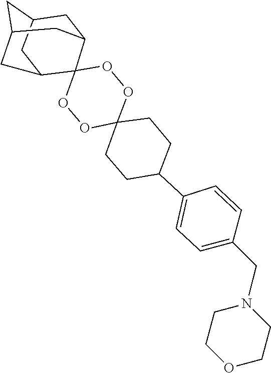 [SMILES: c1cc(C2CCC3(CC2)OOC2(OO3)C3CC4CC(C3)CC2C4)ccc1CN1CCOCC1]

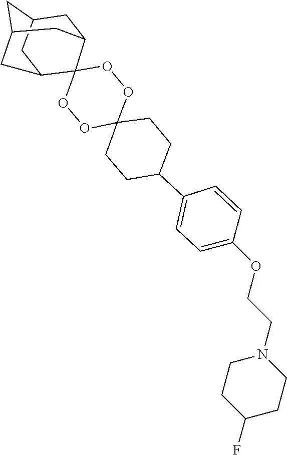 [SMILES: FC1CCN(CCOc2ccc(C3CCC4(CC3)OOC3(OO4)C4CC5CC(C4)CC3C5)cc2)CC1]

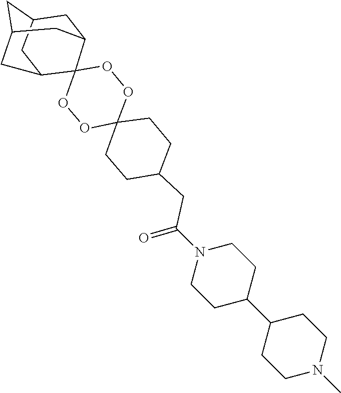 [SMILES: CN1CCC(C2CCN(C(=O)CC3CCC4(CC3)OOC3(OO4)C4CC5CC(C4)CC3C5)CC2)CC1]

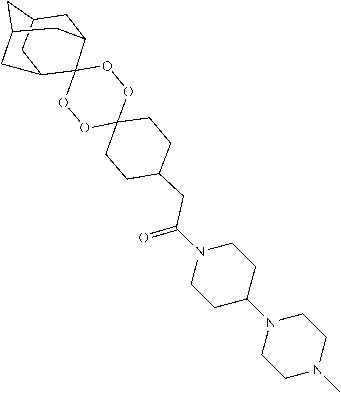 [SMILES: CN1CCN(C2CCN(C(=O)CC3CCC4(CC3)OOC3(OO4)C4CC5CC(C4)CC3C5)CC2)CC1]

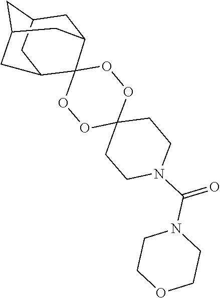 [SMILES: O=C(N1CCOCC1)N1CCC2(CC1)OOC1(OO2)C2CC3CC(C2)CC1C3]

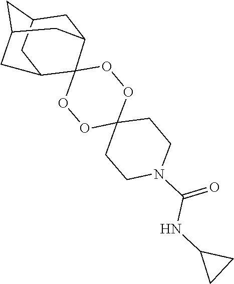 [SMILES: O=C(NC1CC1)N1CCC2(CC1)OOC1(OO2)C2CC3CC(C2)CC1C3]

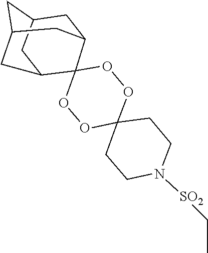 [SMILES: CCS(=O)(=O)N1CCC2(CC1)OOC1(OO2)C2CC3CC(C2)CC1C3]

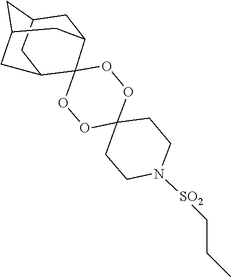 [SMILES: CCCS(=O)(=O)N1CCC2(CC1)OOC1(OO2)C2CC3CC(C2)CC1C3]

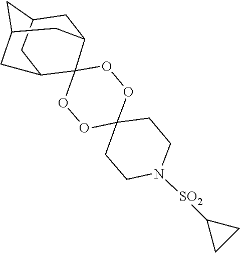 [SMILES: O=S(=O)(C1CC1)N1CCC2(CC1)OOC1(OO2)C2CC3CC(C2)CC1C3]

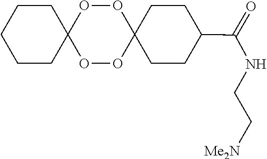 [SMILES: CN(C)CCNC(=O)C1CCC2(CC1)OOC1(CCCCC1)OO2]

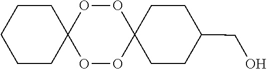 [SMILES: OCC1CCC2(CC1)OOC1(CCCCC1)OO2]

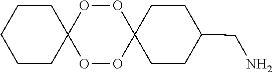 [SMILES: NCC1CCC2(CC1)OOC1(CCCCC1)OO2]

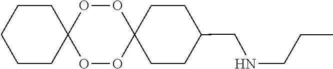 [SMILES: CCCNCC1CCC2(CC1)OOC1(CCCCC1)OO2]

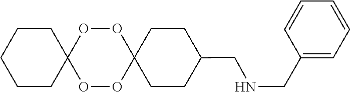 [SMILES: c1ccc(CNCC2CCC3(CC2)OOC2(CCCCC2)OO3)cc1]

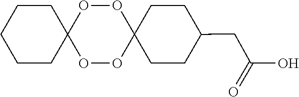 [SMILES: O=C(O)CC1CCC2(CC1)OOC1(CCCCC1)OO2]

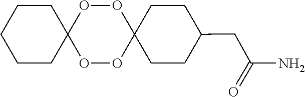 [SMILES: NC(=O)CC1CCC2(CC1)OOC1(CCCCC1)OO2]

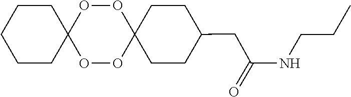 [SMILES: CCCNC(=O)CC1CCC2(CC1)OOC1(CCCCC1)OO2]

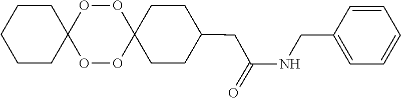 [SMILES: O=C(CC1CCC2(CC1)OOC1(CCCCC1)OO2)NCc1ccccc1]

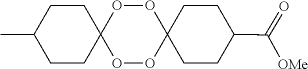 [SMILES: COC(=O)C1CCC2(CC1)OOC1(CCC(C)CC1)OO2]

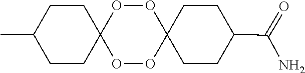 [SMILES: CC1CCC2(CC1)OOC1(CCC(C(N)=O)CC1)OO2]

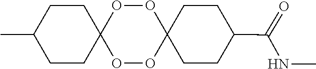

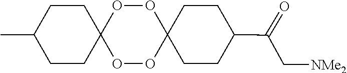 [SMILES: CC1CCC2(CC1)OOC1(CCC(C(=O)CN(C)C)CC1)OO2]

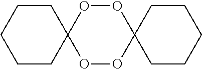 [SMILES: C1CCC2(CC1)OOC1(CCCCC1)OO2]

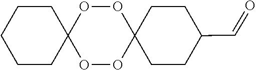 [SMILES: O=CC1CCC2(CC1)OOC1(CCCCC1)OO2]

The compound to be administered, its dose, dosage form, and overall therapeutic regimen are selected by a treating clinician on the basis, in part, of a diagnosis of MDS in a patient. In this regard, MDS can be described by reference to its classification and subtype. Thus, in accordance with some embodiments, one classification of MDS is primary MDS, which is characterized by the absence of any apparent risk factors in a patient. Examples of risk factors include advanced age of patient (MDS is uncommon in patients younger than 50, and most occurrences of MDS are in patients who are at least 70 or 80): sex of the patient (MDS is more common in men); prior cancer treatment, such as chemotherapy, radiation, or both; presence of genetic syndromes such as Fanconi anemia, Shwachman-Diamond syndrome. Diamond Blackfan anemia, Familial platelet disorder with a propensity to myeloid malignancy, Severe congenital neutropenia, and Dyskeratosis congenita; familial MDS; smoking; and environmental exposures, such as radiation from a nuclear reactor accident or atomic bomb blast; and long-term workplace exposure to benzene or other known carcinogens in the chemical or petroleum industries.

Another classification of MDS is secondary MDS, which is often occasioned by damage to the patient's DNA from chemotherapy and/or radiation therapy previously given to the patient for treatment of another medical condition. Secondary MDS can develop 2 to 10 years after such treatment, and is often associated with more complex chromosomal abnormalities.

The MDS also can be characterized, in some embodiments, by reference to its subtype. MDS subtypes include refractory anemia (RA), refractory anemia with ringed sideroblasts (RARS), refractory cytopenia with multilineage dysplasia (RCMD), refractory cytopenia with multilineage dysplasia and ringed sideroblasts (RCMD-RS), refractory anemia with excess blasts (RAEB), myclodysplastic syndrome unclassified (MDS-U), and MDS associated with isolated del(5q).

A subject suffering from MDS also is at risk for disease progression to acute myeloid leukemia (AML). For example, the risk is especially high in subjects who have been diagnosed with RAEB. Thus, in an embodiment, the present disclosure also provides a method for slowing or preventing the development of leukemia, such as AML, in a subject who suffers from MDS, comprising administering to the subject an antimalarial endoperoxide compound or pharmaceutically acceptable salt thereof as described herein. A clinician diagnoses MDS in the subject and, in accordance with accepted criteria, predicts the subject's risk of developing AML. For example, the clinician can utilize the International Prognostic Scoring System (IPSS-R) to assess risk factors: these include the percentage of blasts found in the subject's bone marrow, the type and extent of chromosomal changes, and the levels of hemoglobin found in red blood cells, platelets, and neutrophils. A lower IPSS-R score correlates to a subject with MDS having lower risk for developing AML and a higher overall prognosis for survival, in which case less aggressive treatment is needed. In contrast, a higher IPSS-R score, such as in subjects with a high-risk MDS, can indicate the need for more aggressive treatment.

### Pharmaceutical Composition

The disclosure also provides, optionally for use in combination with the methods described herein, a pharmaceutical composition comprising a therapeutically effective amount of one or more compounds or a pharmaceutically acceptable salt described herein, in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Tables 1 to 4, or a pharmaceutically acceptable salt thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt thereof that is administered is governed by such considerations, and is the minimum amount necessary to restore normal erythropoiesis in patients suffering from MDS, to induce differentiation of MDS-L cells, to inhibit heme biosynthesis, or any combination thereof. Such amount may be below the amount that is toxic to normal cells, or the subject as a whole. Generally, the initial therapeutically effective amount of a compound of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound of the present disclosure. In another embodiment, such dosage forms contain from about 50 mg to about 500 mg of a compound of the present disclosure. In yet another embodiment, such dosage forms contain from about 25 mg to about 200 mg of a compound of the present disclosure. In still another embodiment, such dosage forms contain from about 10 mg to about 100 mg of a compound of the present disclosure. In a further embodiment, such dosage forms contain from about 5 mg to about 50 mg of a compound of the present disclosure. In any of the foregoing embodiments the dosage form can be administered once a day or twice per day.

The compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In an embodiment, also encompassed are pharmaceutical compositions suitable for single unit dosages that comprise a compound of the disclosure or its pharmaceutically acceptable salt, and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of a compound of the present disclosure.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate: granulating and disintegrating agents, for example, corn starch, or alginic acid: binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydroxypropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents.

The pharmaceutical composition may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compound as described herein may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### EXAMPLES

The following non-limiting examples are additional embodiments that further illustrate the present disclosure.

All endoperoxide compounds described in the present disclosure are obtained from commercial sources or are synthesized according to published procedures. See, e.g., M. Rudrapal et al. Drug Design. Development and Therapy 10 (2016) 3575-3590, D. M. Opsenica et al. J. Serb. Chem. Soc. 74(11) (2009) 1155-1193, and references cited therein.

MDS-L differentiation assay. Given a limited number of patient samples and the low yield from each biopsy, it is not tractable to conduct high-throughput screens with primary cells. We therefore obtained a cell line isolated from CD34+MDS HPCs termed MDS-L, which has been previously demonstrated to be differentiation competent when supplemented with appropriate cytokines (J. Fang et al., Nat. Med. 22(7) (2016) 727-734: K. Tohyama et al., Br. J. Haematol. 91(4) (1995) 795-799).

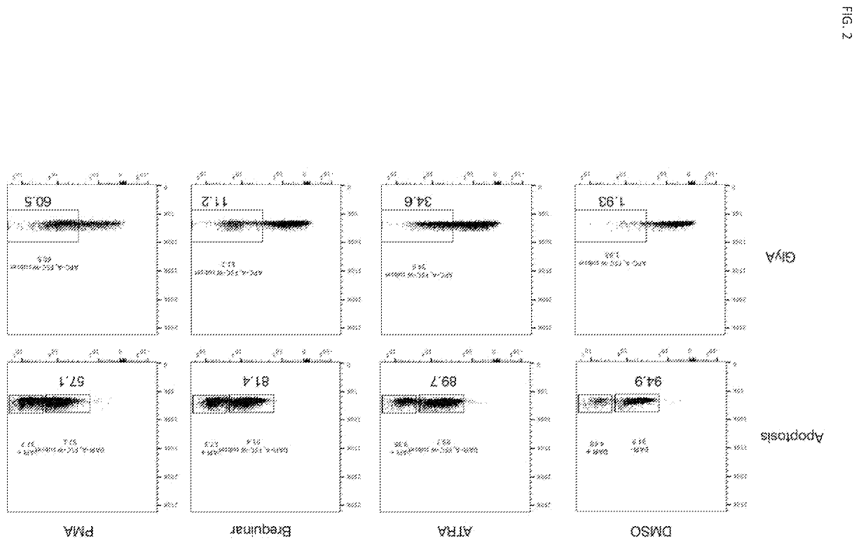

To establish a screening assay, we selected known differentiation inducers of acute myeloid leukemia cells including retinoic acid (ATRA), Brequinar, and phorbol 12-myristate 13-acetate (PMA). We found that all agents were efficacious at inducing differentiation to more terminal blood lineages, a measurement assessed by cellular immunopositivity to anti-GlyA directed antibodies in a 384-well FACS-based assay. Among these, we found ATRA to be highly efficacious at inducing differentiation while displaying minimal cytotoxicity as readout by live dead cell gating (FIG. 2). For these reasons, ATRA served as a suitable positive control for screening efforts. With this established miniaturized assay, we then screened the comprehensive drug repurposing library ReFRAME for previously unannotated agents that induce differentiation of MDS-L cells with minimal cytotoxicity. Among the top validated hits from this screen were molecules representative of known mechanisms for inducing cell cycle arrest in MDS cells including topoisomerase inhibitors, tubulin-targeting agents, DNA damaging chemotherapeutics, and a number of receptor tyrosine kinase inhibitors, among others.

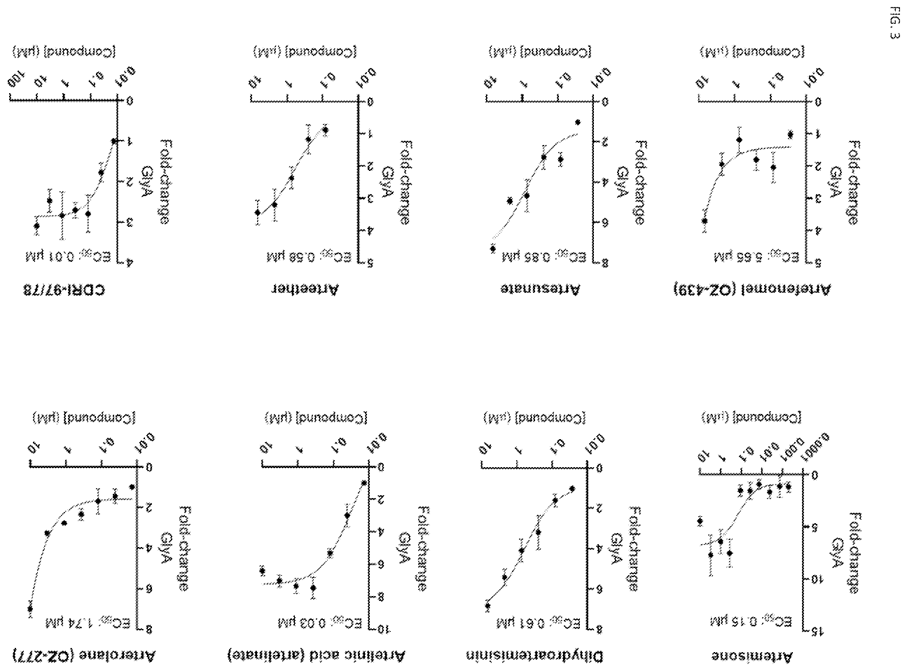

Surprisingly, screening hits with no previously reported activity in MDS biology were two semi-synthetic derivatives of artemisinin, a naturally occurring endoperoxide that is an established antimalarial drug. We therefore evaluated representative endoperoxides, including dihydroartemisinin, arteether, artemisone, artesunate, and the synthetic trioxolane artefenomel. MDS-L were cultured in RPMI (17-105-CV, Corning) without phenol red and supplemented with 2 mM glutamine and 10% FBS. Cells first expanded in IL-3 until 70% confluence then pre-conditioned for 14 days with IL-3 (200-03, PeproTech), GCSF (300-23, PeproTech), and EPO (287-TC, R&D Systems) to promote erythroid and myeloid differentiation. A density of 10000 cells per well were used in phenotypic assays. MDSL cells were spun and treatment media was replaced with media containing APC Anti-CD235a (GlyA) (551336. BD Biosciences) (1.25 μL per 50 μL test) and incubated for 1 hour at 4° C. Cells were washed then counter stained with SYTOX-Green 1 μM per 100 μL prior to flow cytometry, analysis. Dose response and pharmacological assays were analyzed using a ZE5 Cell Analyzer (BioRad) equipped with Everest software. Single, live, cells were analyzed for GlyA expression using FlowJo v10.7.2 software. Results are shown in Table 5. All the tested compounds were efficacious at inducing GlyA positivity induction in MDS-L cells; the half maximal potencies of these agents varied over two orders of magnitude (FIG. 3).

TABLE 5
MDS-L differentiation assay of antimalarial endoperoxide compounds.

| | | MDS-L | |
|---|---|---|---|
| | | GlyA | Maximal |
| | | induction | MDS- |
| Compound | | EC₅₀ | LGlyA fold |
| name | Structure | (μM) | induction |
| Artelinic acid (artelinate) | 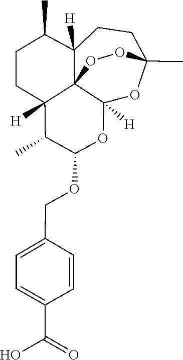 [SMILES: C[C@H]1[C@@H](OCc2ccc(C(=O)O)cc2)O[C@@H]2O[C@@]3(C)CC[C@H]4[C@H](C)CC[C@@H]1[C@@]24OO3] | 0.03 | 7.26 |
| Artemisone | 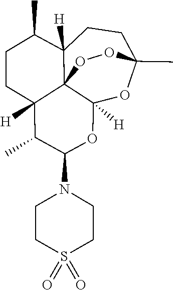 [SMILES: C[C@H]1[C@H](N2CCS(=O)(=O)CC2)O[C@@H]2O[C@@]3(C)CC[C@H]4[C@H](C)CC[C@@H]1[C@@]24OO3] | 0.15 | 6.84 |
| Arteether | 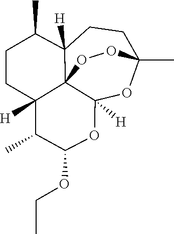 [SMILES: CCO[C@H]1O[C@@H]2O[C@@]3(C)CC[C@H]4[C@H](C)CC[C@@H]([C@H]1C)[C@@]24OO3] | 0.58 | 3.82 |
| Dihydroarte misin | 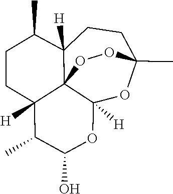 [SMILES: C[C@H]1[C@@H](O)O[C@@H]2O[C@@]3(C)CC[C@H]4[C@H](C)CC[C@@H]1[C@@]24OO3] | 0.61 | 7.09 |
| Artesunate | 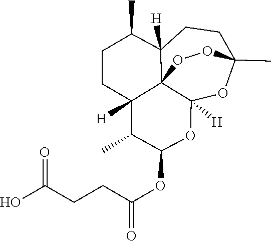 [SMILES: C[C@H]1[C@H](OC(=O)CCC(=O)O)O[C@@H]2O[C@@]3(C)CC[C@H]4[C@H](C)CC[C@@H]1[C@@]24OO3] | 0.85 | 7.51 |
| Arterolane (OZ-277) | 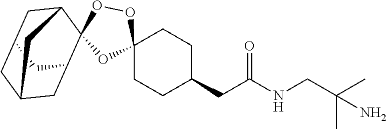 [SMILES: CC(C)(N)CNC(=O)C[C@H]1CC[C@]2(CC1)OO[C@]1(O2)[C@@H]2C[C@H]3C[C@@H](C2)C[C@@H]1C3] | 1.74 | 7.02 |
| Artefenomel (OZ-439) | 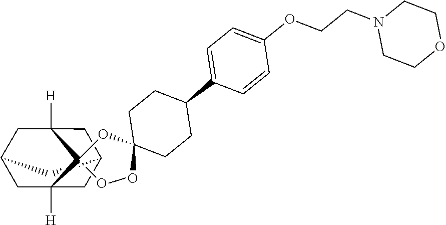 | 5.65 | 3.71 |
| CDRI-97/78 | 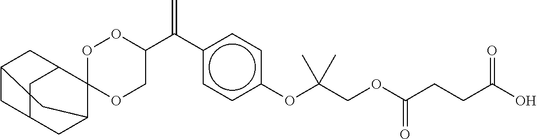 | 0.01253 | 3.09 |

Likewise, we demonstrated that the exemplary artemisinin compounds aretmisonc and arteether efficaciously induced terminal differentiation of MDS-L cells to CFU-Es while decreasing the number of BFU-E. CFU-GM and CFU-GEMM type colonies in CFU assays. Additionally, artemisone and arteether efficaciously promoted the loss of CD71 and displayed no significant induction in apoptosis below 20 μM. Together, these results indicate a functional and non-toxic impact on multi-lineage differentiation in MDS blastic cells. In contrast, the non-peroxide containing molecule 2-deoxy-artemisinin was inactive in differentiation assays.

Heme depletion induces terminal differentiation of MDS-L cells. The purpose of this example is to establish a pharmacological understanding of how endoperoxides, which have no known therapeutic activity in MDS, are surprisingly efficacious for inducing the differentiation of MDS blasts. Because 2-deoxy-artemisinin is inactive for this purpose, we reasoned that the endoperoxide core of the active molecules identified in the screen corresponds to their activity, indicating a similar mechanism to how artemisinins induce compromise of the malarial parasite. In this therapeutically distinct context, artemisinins are known to be activated by heme-iron, a catalytic event which catalyzes cleavage of the endoperoxide, generating free radicals and forming reactive species that can inactivate essential proteins required for parasite survival.

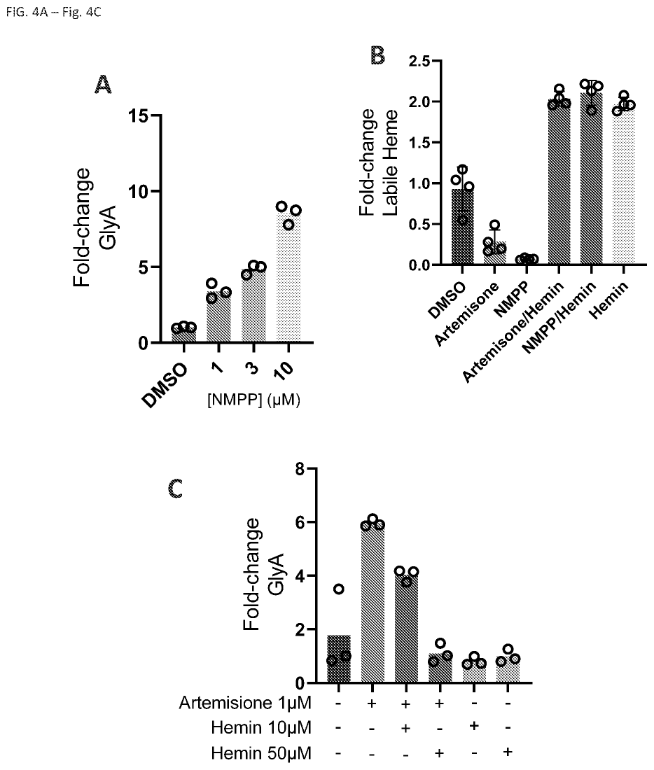

An additional consequence of this mechanism is the alkylation of artemisinin to heme, resulting in its functional inactivation. Because we found no other reactive oxygen species (ROS)-inducers or alkylators as active hits from the screen, we reasoned that heme depletion is a way in which artemisinins promote differentiation of this cellular population. To illustrate further, we found that inhibition of heme biosynthesis effectively induced MDS differentiation, as treatment with the ferrochelatase inhibitor NMPP (N-methylprotoporphyrin) was found to increase GlyA positivity in MDS-L cells (FIG. 4A). Similarly, we found that treatment with NMPP and representative artemisinin derivative artemisone efficiently reduced levels of labile heme in MDS-L cells over relevant time periods (FIG. 4B). Further, we found that the addition of exogenous heme in the form of hemin suppressed the potential of artemisone to induce differentiation of MDS-L cells (FIG. 4C). These results position pharmacological heme depletion, such as by direct conjugation or by inhibition of its biosynthesis, as a means to induce differentiation of an MDS relevant cell type and a means for differentiation-inducing therapy for MDS.

We claim:

1. A method for treating a myelodysplastic syndrome (MDS) in a subject suffering therefrom, comprising administering to the subject a therapeutically effective amount of an antimalarial endoperoxide compound or a pharmaceutically acceptable salt thereof.

2. A method for slowing or preventing the development of leukemia in a subject, comprising administering to the subject a therapeutically effective amount of an antimalarial endoperoxide compound or a pharmaceutically acceptable salt thereof, wherein the subject is one who suffers from a myelodysplastic syndrome (MDS).

3. The method according to claim 2, wherein the leukemia is acute myeloid leukemia (AML).

4. The method according to any one of claims 1 to 3, wherein the endoperoxide compound is chosen from artemisinin and derivatives thereof, a compound containing a 1,2,4-trioxane ring, a compound containing a 1,2,4-trioxolane ring, a compound containing a 1,2,4,5-tetraoxane ring, and pharmaceutically acceptable salts thereof.

5. The method according to any one of claims 1 to 4, wherein the endoperoxide is chosen from artemisinin and derivatives thereof, or a pharmaceutically acceptable salt thereof.

6. The method according to claim 5, wherein the artemisinin and derivatives thereof or a pharmaceutically acceptable salt thereof is one selected from the following table: 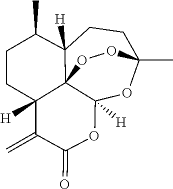 [SMILES: C=C1C(=O)O[C@@H]2O[C@@]3(C)CC[C@H]4[C@H](C)CC[C@@H]1[C@@]24OO3]
artemisinin 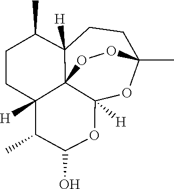
dihydroartemisinin 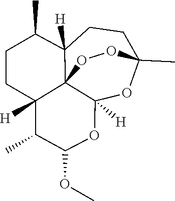
artemether 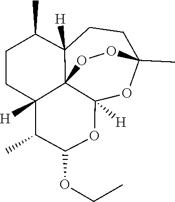
arteether 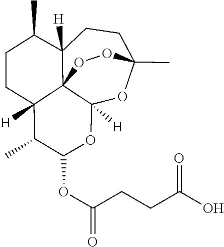
artesunate 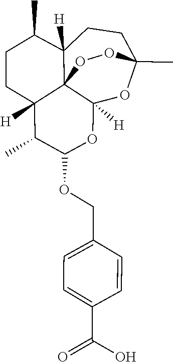
artelinate 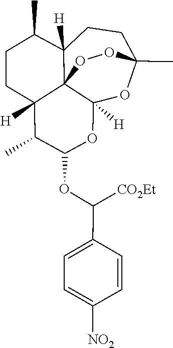 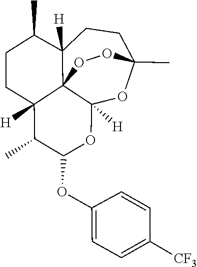 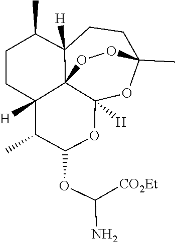 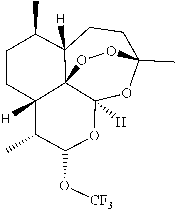 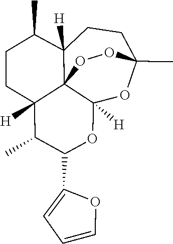 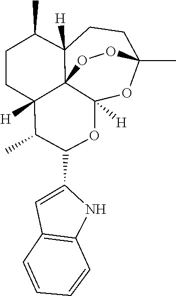 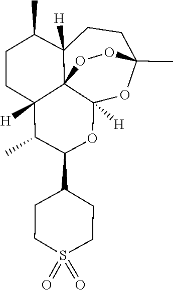 [SMILES: C[C@H]1[C@H](C2CCS(=O)(=O)CC2)O[C@@H]2O[C@@]3(C)CC[C@H]4[C@H](C)CC[C@@H]1[C@@]24OO3]
Artemisone 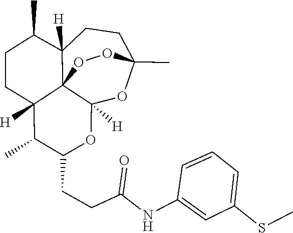
3-artesanilide

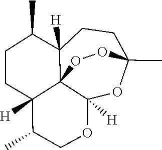 [SMILES: C[C@@H]1CC[C@H]2[C@@H](C)CO[C@@H]3O[C@@]4(C)CC[C@@H]1[C@]32OO4]

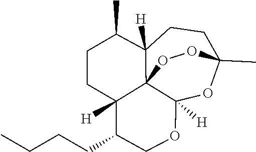 [SMILES: CCCC[C@H]1CO[C@@H]2O[C@@]3(C)CC[C@H]4[C@H](C)CC[C@@H]1[C@@]24OO3]

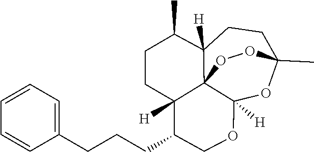 [SMILES: C[C@@H]1CC[C@H]2[C@@H](CCCc3ccccc3)CO[C@@H]3O[C@@]4(C)CC[C@@H]1[C@]32OO4]

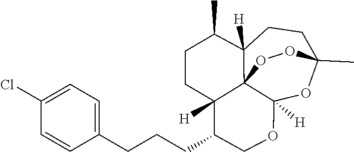 [SMILES: C[C@@H]1CC[C@H]2[C@@H](CCCc3ccc(Cl)cc3)CO[C@@H]3O[C@@]4(C)CC[C@@H]1[C@]32OO4]

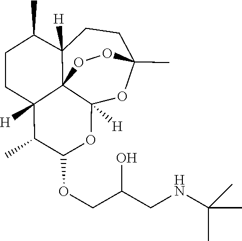

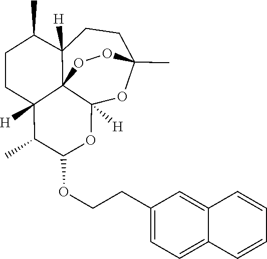

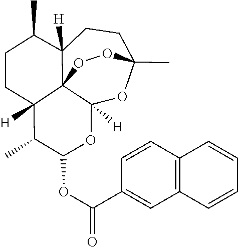

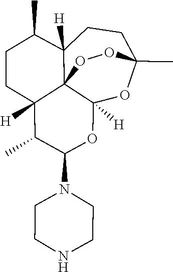 [SMILES: C[C@H]1[C@H](N2CCNCC2)O[C@@H]2O[C@@]3(C)CC[C@H]4[C@H](C)CC[C@@H]1[C@@]24OO3]

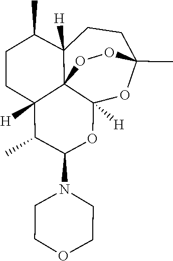 [SMILES: C[C@H]1[C@H](N2CCOCC2)O[C@@H]2O[C@@]3(C)CC[C@H]4[C@H](C)CC[C@@H]1[C@@]24OO3]

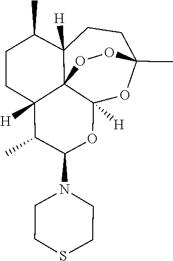 [SMILES: C[C@H]1[C@H](N2CCSCC2)O[C@@H]2O[C@@]3(C)CC[C@H]4[C@H](C)CC[C@@H]1[C@@]24OO3]

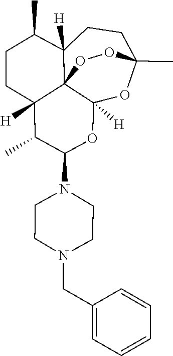 [SMILES: C[C@H]1[C@H](N2CCN(Cc3ccccc3)CC2)O[C@@H]2O[C@@]3(C)CC[C@H]4[C@H](C)CC[C@@H]1[C@@]24OO3]

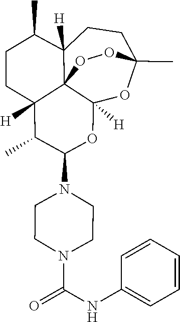 [SMILES: C[C@H]1[C@H](N2CCN(C(=O)Nc3ccccc3)CC2)O[C@@H]2O[C@@]3(C)CC[C@H]4[C@H](C)CC[C@@H]1[C@@]24OO3]

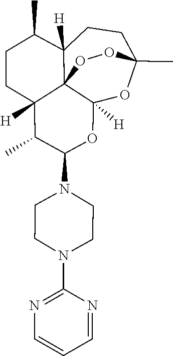 [SMILES: C[C@H]1[C@H](N2CCN(c3ncccn3)CC2)O[C@@H]2O[C@@]3(C)CC[C@H]4[C@H](C)CC[C@@H]1[C@@]24OO3]

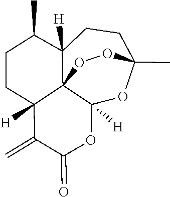 [SMILES: C=C1C(=O)O[C@@H]2O[C@@]3(C)CC[C@H]4[C@H](C)CC[C@@H]1[C@@]24OO3]

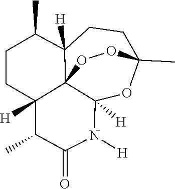 [SMILES: C[C@@H]1CC[C@H]2[C@@H](C)C(=O)N[C@@H]3O[C@@]4(C)CC[C@@H]1[C@]32OO4]

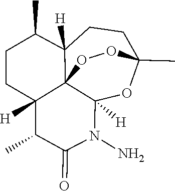 [SMILES: C[C@@H]1CC[C@H]2[C@@H](C)C(=O)N(N)[C@@H]3O[C@@]4(C)CC[C@@H]1[C@@]23OO4]

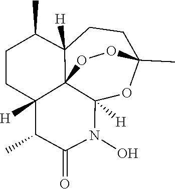 [SMILES: C[C@@H]1CC[C@H]2[C@@H](C)C(=O)N(O)[C@@H]3O[C@@]4(C)CC[C@@H]1[C@@]23OO4]

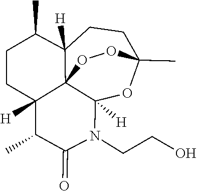 [SMILES: C[C@@H]1CC[C@H]2[C@@H](C)C(=O)N(CCO)[C@@H]3O[C@@]4(C)CC[C@@H]1[C@@]23OO4]

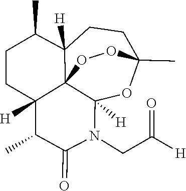 [SMILES: C[C@@H]1CC[C@H]2[C@@H](C)C(=O)N(CC=O)[C@@H]3O[C@@]4(C)CC[C@@H]1[C@@]23OO4]

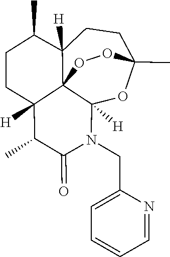 [SMILES: C[C@@H]1CC[C@H]2[C@@H](C)C(=O)N(Cc3ccccn3)[C@@H]3O[C@@]4(C)CC[C@@H]1[C@@]23OO4]

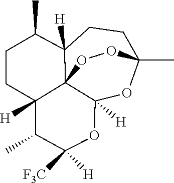 [SMILES: C[C@H]1[C@H](C(F)(F)F)O[C@@H]2O[C@@]3(C)CC[C@H]4[C@H](C)CC[C@@H]1[C@@]24OO3]

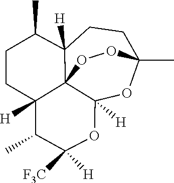 [SMILES: C[C@H]1[C@H](C(F)(F)F)O[C@@H]2O[C@@]3(C)CC[C@H]4[C@H](C)CC[C@@H]1[C@@]24OO3]

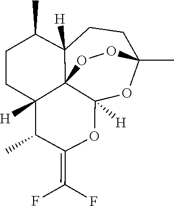 [SMILES: C[C@@H]1CC[C@H]2[C@@H](C)C(=C(F)F)O[C@@H]3O[C@@]4(C)CC[C@@H]1[C@]32OO4]

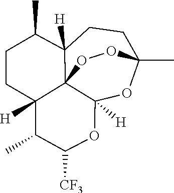

7. The method according to any one of claims 1 to 4, wherein the endoperoxide is a compound containing a 1,2,4-trioxane ring, or a pharmaceutically acceptable salt thereof.

8. The method according to claim 7, wherein the compound containing a 1,2,4-trioxane ring, or a pharmaceutically acceptable salt thereof, is one selected from the following table:

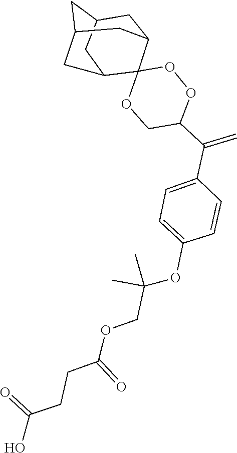 [SMILES: C=C(c1ccc(OC(C)(C)COC(=O)CCC(=O)O)cc1)C1COC2(OO1)C1CC3CC(C1)CC2C3]

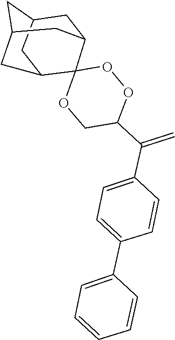 [SMILES: C=C(c1ccc(-c2ccccc2)cc1)C1COC2(OO1)C1CC3CC(C1)CC2C3]

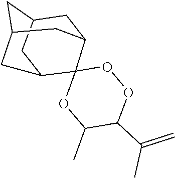 [SMILES: C=C(C)C1OOC2(OC1C)C1CC3CC(C1)CC2C3]

9. The method according to any one of claims 1 to 4, wherein the endoperoxide is a compound containing a 1,2,4-trioxolane ring, or a pharmaceutically acceptable salt thereof.

10. The method according to claim 9, wherein the compound containing a 1,2,4-trioxolane ring, or a pharmaceutically acceptable salt thereof, is one selected from the following table: 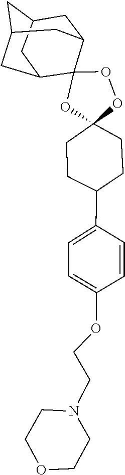
artefenomel

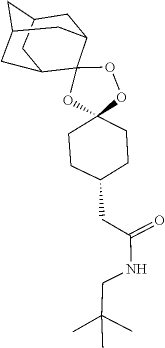

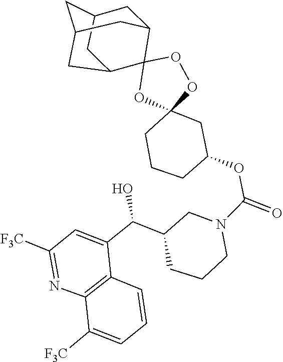

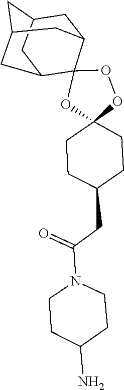

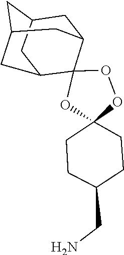

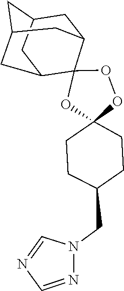

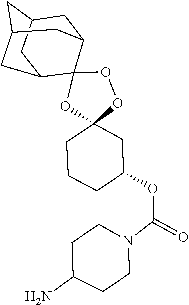

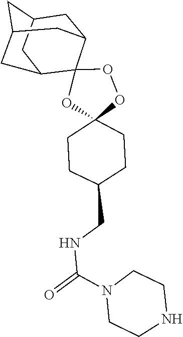

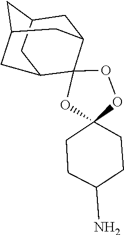

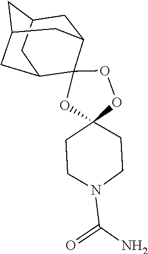

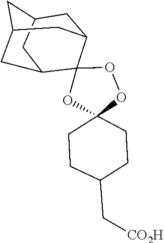

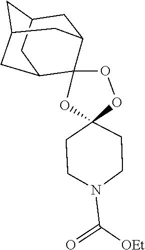

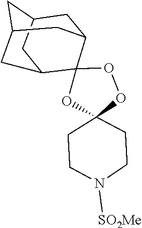

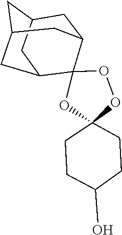

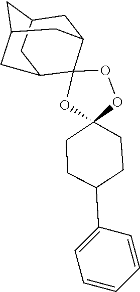

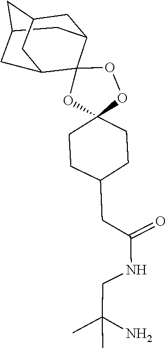

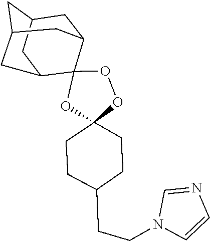

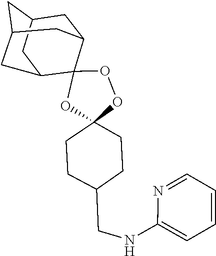

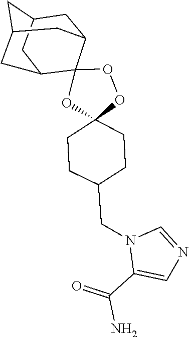

11. The method according to any one of claims 1 to 4, wherein the endoperoxide is a compound containing a 1,2,4,5-tetraoxane ring, or a pharmaceutically acceptable salt thereof.

12. The method according to claim 11, wherein the compound containing a 1,2,4,5-tetraoxane ring, or a pharmaceutically acceptable salt thereof, is one selected from the following table:

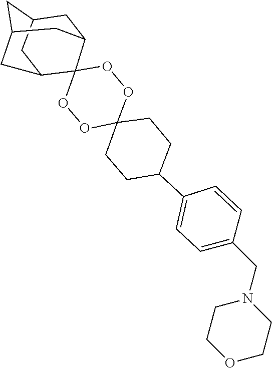 [SMILES: c1cc(C2CCC3(CC2)OOC2(OO3)C3CC4CC(C3)CC2C4)ccc1CN1CCOCC1]

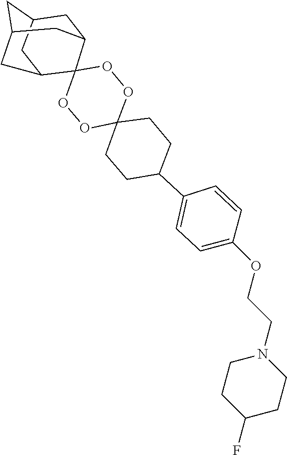 [SMILES: FC1CCN(CCOc2ccc(C3CCC4(CC3)OOC3(OO4)C4CC5CC(C4)CC3C5)cc2)CC1]

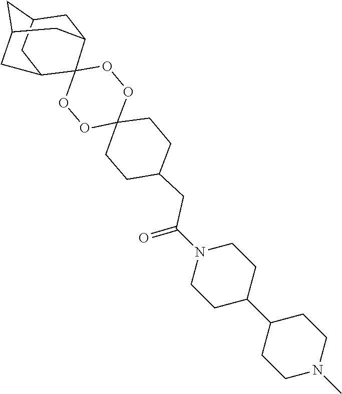 [SMILES: CN1CCC(C2CCN(C(=O)CC3CCC4(CC3)OOC3(OO4)C4CC5CC(C4)CC3C5)CC2)CC1]

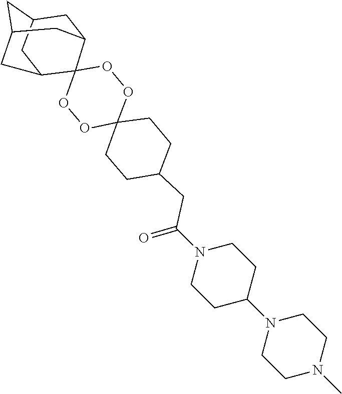 [SMILES: CN1CCN(C2CCN(C(=O)CC3CCC4(CC3)OOC3(OO4)C4CC5CC(C4)CC3C5)CC2)CC1]

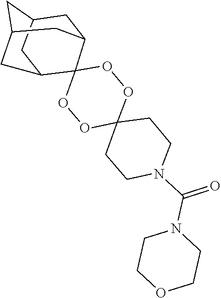 [SMILES: O=C(N1CCOCC1)N1CCC2(CC1)OOC1(OO2)C2CC3CC(C2)CC1C3]

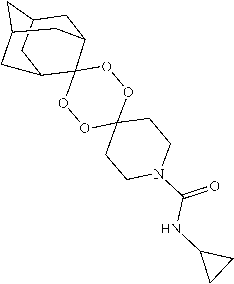 [SMILES: O=C(NC1CC1)N1CCC2(CC1)OOC1(OO2)C2CC3CC(C2)CC1C3]

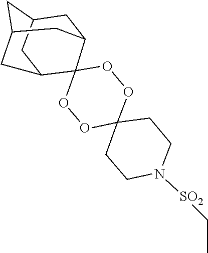 [SMILES: CCS(=O)(=O)N1CCC2(CC1)OOC1(OO2)C2CC3CC(C2)CC1C3]

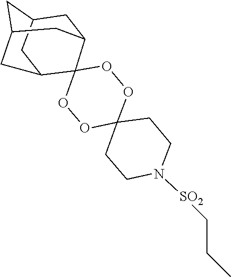 [SMILES: CCCS(=O)(=O)N1CCC2(CC1)OOC1(OO2)C2CC3CC(C2)CC1C3]

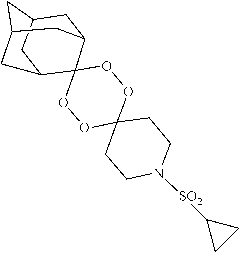 [SMILES: O=S(=O)(C1CC1)N1CCC2(CC1)OOC1(OO2)C2CC3CC(C2)CC1C3]

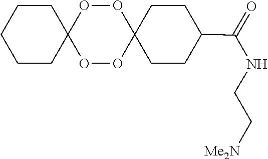 [SMILES: CN(C)CCNC(=O)C1CCC2(CC1)OOC1(CCCCC1)OO2]

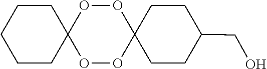 [SMILES: OCC1CCC2(CC1)OOC1(CCCCC1)OO2]

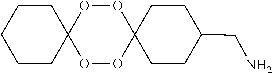 [SMILES: NCC1CCC2(CC1)OOC1(CCCCC1)OO2]

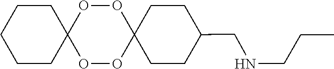 [SMILES: CCCNCC1CCC2(CC1)OOC1(CCCCC1)OO2]

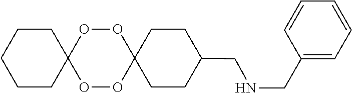 [SMILES: c1ccc(CNCC2CCC3(CC2)OOC2(CCCCC2)OO3)cc1]

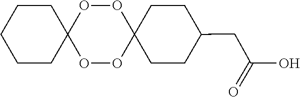 [SMILES: O=C(O)CC1CCC2(CC1)OOC1(CCCCC1)OO2]

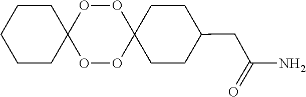 [SMILES: NC(=O)CC1CCC2(CC1)OOC1(CCCCC1)OO2]

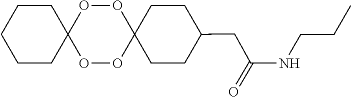 [SMILES: CCCNC(=O)CC1CCC2(CC1)OOC1(CCCCC1)OO2]

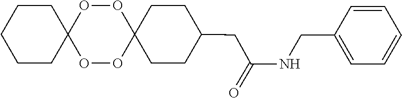 [SMILES: O=C(CC1CCC2(CC1)OOC1(CCCCC1)OO2)NCc1ccccc1]

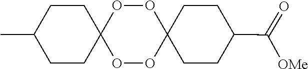 [SMILES: COC(=O)C1CCC2(CC1)OOC1(CCC(C)CC1)OO2]

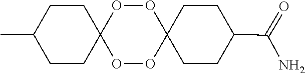 [SMILES: CC1CCC2(CC1)OOC1(CCC(C(N)=O)CC1)OO2]

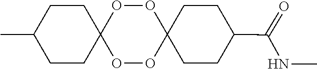

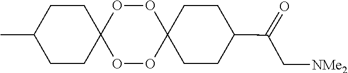 [SMILES: CC1CCC2(CC1)OOC1(CCC(C(=O)CN(C)C)CC1)OO2]

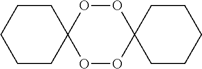 [SMILES: C1CCC2(CC1)OOC1(CCCCC1)OO2]

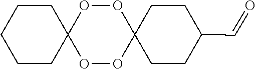 [SMILES: O=CC1CCC2(CC1)OOC1(CCCCC1)OO2]

13. The method according to any one of claims 1 to 12, wherein the MDS is primary MDS.

14. The method according to any one of claims 1 to 12, wherein the MDS is secondary MDS.

15. The method according to any one of claims 1 to 14, wherein the MDS is one or more subtypes chosen from refractory anemia (RA), refractory anemia with ringed sideroblasts (RARS), refractory cytopenia with multilineage dysplasia (RCMD), refractory cytopenia with multilineage dysplasia and ringed sideroblasts (RCMD-RS), refractory anemia with excess blasts (RAEB), myelodysplastic syndrome unclassified (MDS-U), and MDS associated with isolated del(5q).
